(19) United States
(12) Patent Application Publication
(10) Pub. No.: US 2022/0049172 A1
(43) Pub. Date: Feb. 17, 2022

(54) PROCESS FOR CATALYTIC PRODUCTION OF PROPANOL

(71) Applicant: Neste Oyj, Espoo (FI)

(72) Inventors: Marja TIITTA, Porvoo (FI); Anna KARVO, Porvoo (FI); Ulla KIISKI, Porvoo (FI); Terhi KOLEHMAINEN, Porvoo (FI); Jukka MYLLYOJA, Porvoo (FI); Ville PAASIKALLIO, Porvoo (FI)

(73) Assignee: Neste Oyj, Espoo (FI)

(21) Appl. No.: 17/418,349

(22) PCT Filed: Dec. 20, 2019
(86) PCT No.: PCT/FI2019/050914

§ 371 Date: Jun. 25, 2021

(30) Foreign Application Priority Data
Dec. 31, 2018 (FI) 20186145

(51) Int. Cl. C10L 1/02; C11B 7/00; C07C 29/60; C07C 29/84; C10L 1/182; C10G 3/00; C07C 45/29; C10G 45/64; C10G 45/62

(52) U.S. Cl. CPC C10L 1/023; C10L 2290/24; C07C 29/60; C07C 29/84; C10L 1/1824; C10G 3/50; C07C 45/29; C10G 45/64; C10G 45/62; C10G 3/46; C10L 2200/0469; C10G 2300/1011; C10L 2200/0423; C10G 2400/02; C10L 2270/023; C10G 2300/4006; C10G 2300/4012; C10G 2300/4018; C10G 2400/08; C10L 2200/043; C10L 2270/04; C10L 2290/543; C11B 7/00

(57) ABSTRACT

The present disclosure is related to a multistep process for producing renewable gasoline components from a glyceride containing feedstock. The glycerides are split to provide a stream containing fatty acids, or esters of fatty acids, and another stream containing glycerol and water. Glycerol, preferably as crude glycerol recovered from splitting, is next converted to propanols at vapor phase, providing a renewable propanol gasoline component. Another renewable gasoline component is obtained from hydroprocessing of the fatty acids or esters thereof, as a renewable paraffinic naphtha component. Blending the renewable components can provide a novel 100% renewable gasoline.

### FIELD OF THE INVENTION

The present disclosure relates to renewable fuel component production and renewable base oil production. As part of the overall process, a process comprising conversion of glycerol to alcohols is disclosed. Further, combined production of renewable propanol and renewable fuel components is disclosed. As one embodiment a novel, 100% renewable gasoline is provided. Furthermore, a method is provided relating to more efficient glyceride containing feedstock material utilization in renewable fuel and base oil production.

### BACKGROUND OF THE INVENTION

In the prior art processes where feeds comprising triglycerides are converted to paraffinic fuels, very little attention has been paid to C3-sceleton originating from glycerol in triglycerides. Typically, when fatty acid residues have been converted to higher carbon number alkanes providing paraffinic fuel components, said C3 stream has entered hydrogen rich processing conditions as well, and has thereby been converted to propane, and possibly removed from the process. However, as such, gaseous propane C3 is not among the most desired hydrocarbon products. Further, since the glyceridic C3-sceleton present in triglycerides has high relative oxygen content, its reduction to the alkane consumes in vain a portion of the available hydrogen and thus hydrogenation capacity.

Hydrodeoxygenation is a well-established process for fuel production. But when applied to feedstock originating from renewable sources, it does not utilize the natural characteristics of the feedstock in most efficient way. For example, reduction of triglycerides into paraffinic hydrocarbons involves saturation of C═C double bonds and loss of all oxygen-containing functionalities even though they could be useful and valuable for certain product fractions. Therefore, there is a need for more sophisticated overall processes, wherein feedstock characteristics are better taken into consideration and used. Further, there is a need for decreasing or at least avoiding excessive hydrogen consumption. Yet, there is a need to minimize oxygen-containing compounds ending up in low value products.

Fuels, especially transportation fuels, are strictly controlled and highly standardized products. Standards, such as EN590 for diesel fuel, EN15940 for paraffinic diesel fuel, EN228 for gasoline, and ASTMD7566 for aviation fuel define ranges within which different types of gasoline, aviation fuel and diesel fuel respectively should fall. Traditionally, transportation fuels have been obtained as distillation cuts or blends thereof originating from crude oil, also referred to as fossil or mineral oils. Concerns about climate change and urge to decrease greenhouse gas emissions have during past decades driven towards replacement of at least part of the fossil energy with renewable blend components. Among very common blends are ethanol-containing gasolines, such as E10, which is a blend of a fossil cut and bio-ethanol fulfilling specifications set to gasolines. Nevertheless, there is a continuous need to find further renewable blend components, preferably contributing to the properties of final product in a positive way.

### SUMMARY OF THE INVENTION

To overcome at least some of the problems of the prior art, herein is provided a novel process for producing renewable fuel components, said process comprising the steps of
a. providing a glyceride containing feedstock; and
b. splitting said glyceride containing feedstock to provide a first stream comprising fatty acids, or esters of fatty acids, and a second stream comprising glycerol and water; and
c. subjecting said second stream obtained from step b, to
   i. at least one evaporation in the presence of 5-90%-wt water of the total second stream weight, wherefrom the vapor phase is directed to;
   ii. catalytic conversion of glycerol to 1-propanol, 2-propanol or a mixture thereof at vapor phase in presence of water and hydrogen, and
   iii. separation and recovery of 1-propanol, 2-propanol or a mixture thereof as a renewable propanol gasoline component;
d. subjecting said first stream to hydroprocessing, to provide a first product stream of renewable paraffinic fuel components comprising i-paraffins and n-paraffins, and
e. separating said first product stream, and recovering renewable fuel components comprising at least one renewable paraffinic naphtha component.

The underlying idea behind the process of the present invention is production of two renewable gasoline components from one glyceride containing feedstock within one process.

Said process provides advantages through efficient conversion of the glyceride containing feedstock into valuable fuel, especially into gasoline, components.

As another aspect, herein is provided gasoline comprising a renewable propanol gasoline component. The renewable propanol contributes to the gasoline composition as an oxygenate. Further, propanols provide a very good octane component, because other renewable paraffinic gasoline components have typically lower octanes.

Further, here is provided a use of a glyceride containing feedstock for production of a renewable propanol gasoline component. Such use allows recovery of glycerol from the feedstock for production of a renewable oxygenate component as a part of complete feedstock utilization. This also contributes to the yield of fuels, since said yield is better due to oxygen present in the gasoline component.

As one further aspect of the present disclosure, here is provided a process for converting glycerol to 1-propanol, 2-propanol or a mixture thereof, by a catalytic conversion at a vapor phase in the presence of water at a pressure between 0.2 and 1.5 MPa, preferably from 0.5 to 1.0 MPa, in the presence of catalyst comprising hydrogenating catalyst, such as metal on an acid oxide support. Said glycerol is provided as a feed comprising glycerol and 5-90%-wt of water of the total feed weight subjected to evaporation wherefrom a vapor phase is conducted to said catalytic conversion. This process provides benefits through impurity removal and enables use of side streams comprising glycerol as a feedstock.

As explained in detail below, further advantages are obtainable through embodiments of the processes and uses briefly outlined here.

### BRIEF DESCRIPTION OF THE DRAWINGS

The invention will be described in more detail by means of preferred embodiments. Reference is made to the following figures, in which

FIG. 1 shows a schematic arrangement of an embodiment of the present process as a flowchart following the stepwise structure of the process of claim 1,

FIG. 2 shows a schematic arrangement of another embodiment of the present process as a flowchart following the stepwise structure of the process of claim 2.

### DETAILED DESCRIPTION OF THE INVENTION

Glycerol (also called glycerine or glycerin) is a simple polyol compound having chemical formula C₃H₈O₃. Hence, with regard to carbon skeleton, it is a C3-derivative, a C3 triol or sugar alcohol. In purified form it is a colorless, odorless, viscous liquid. The glycerol backbone is found in all lipids known as triglycerides, which chemically are esters of glycerol with long-chain carboxylic acids. Due to its sweet-tasting and non-toxic character, it is widely used in the food industry as a sweetener and humectant and in pharmaceutical formulations. Glycerol has three hydroxyl groups contributing to its solubility in water, to its hygroscopic nature and as consequence, to the processing thereof.

As used herein, “crude glycerol” refers to a stream comprising glycerol and typically water. In addition to glycerol, crude glycerol stream may comprise different components, some regarded as impurities, depending on the source of said crude glycerol. Options for further utilization of crude glycerol comprise multistep purification for use in sophisticated applications, where high purity is of essence. Crude glycerol grades commercially available contain from about 80%-wt to 85%-wt glycerol, the application of which nearly always requires further refining. The sweet water from glyceride hydrolysis contains typically up to 20%-wt of glycerol in water. The challenges with some grades of crude glycerol related to high water content, different impurities therein and cost of prior art purification methods can be avoided by the present process.

The sources for crude glycerol may comprise processes wherein glycerol is formed or released as a side product. From biological sources, streams comprising glycerol typically originate from triglyceride materials and refining processes thereof. One widely used industrial process is transesterification of vegetable oils, or fats, with methanol for the fatty acid methyl ester (FAME) production for use as the primary bio source in biodiesel (diesel fuel including a biooxygenate). FAMEs are typically produced by an alkali-catalyzed reaction between fats and methanol in the presence of a base such as sodium hydroxide, sodium methoxide or potassium hydroxide. In FAME production, when glycerol is formed as side product, said crude glycerol obtained includes methanol, soap, and some water as impurities.

Glycerol chemistry offers numerous options for conversion reactions. Considering reductive conversions alone, the three hydroxyl groups provide a range of variables. C3-derivatives with two remaining hydroxyl groups, i.e. propanediols, both 1,2-propanediol and 1,3-propanediol, are used as solvents and additives in many fields such as polymers, cosmetics and pharmaceuticals.

As considered herein, the glycerol conversion is directed to production of propanols. With “propanols” is herein referred to 1-propanol, 2-propanol or a mixture thereof. In some embodiments, it may be desirable to provide 1-propanol as the main product, with 2-propanol present only as a side product, or vice versa. Typically, a mixture of 1-propanol and 2-propanol in any proportion qualifies as a renewable propanol gasoline component and may be referred to as “propanols”. The present inventors have found that the propanol fraction recovered from conversion by distillation or other separation means, typically comprises both 1-propanol and 2-propanol as well as minor amounts of other compounds, such as methanol, acetone, acetol, ethanol and propanediols, i.e. 1,2-propanediol, 1,3-propanidiol. These impurities are present as an amount of less than 5%-wt. Another characteristic for said propanol fraction is the ratio of 2-propanol to 1-propanol.

As used herein, renewable propanol gasoline component refers to propanols produced from 100% renewable glyceride containing feedstock, and hence said propanol provides a 100% biobased component for use in gasoline blends. When calculating the biocontent of a gasoline comprising both fossil and renewable components, said propanols can fully be counted to the non-fossil part.

Conventionally hydrogenolysis of glycerol has been conducted at the liquid phase. Challenge with these conversions is, should the catalyst be at the same phase it might tend to leach out of the reaction. The same applies to some heterogenous catalysts used at liquid phase as well. On the other hand, one problem often associated with known vapor phase processes has been fluctuation and instability of the glycerol. Further, most glycerol conversion processes have been reported using substantially pure glycerol or pure glycerol/water mixtures. Purification of glycerol for fuel production is not feasible. The present inventors have surprisingly found that the process comprising presence of water vapor preferably at pressurized conditions improves thermal stability of glycerol. Water vapor enhances glycerol vaporization. Thereby spontaneous degradation of glycerol at high temperatures can be decreased.

According to the present process, conversion of glycerol-containing stream, which in the overall process is referred to as “second stream”, may be characterized by steps of
at least one evaporation in the presence of water, wherefrom the vapor phase is directed to;
catalytic conversion of glycerol to 1-propanol, 2-propanol or a mixture thereof at vapor phase in presence of water and hydrogen, and
separation and recovery of 1-propanol, 2-propanol or a mixture thereof as a renewable propanol gasoline component.

Glycerol and water, such as crude glycerol, or aqueous glycerol, are fed to the process together, typically via one input line. The first step is evaporation, preferably at 0.5-1 MPa. After evaporation, the formed vapor phase comprises both glycerol and water vapors. For the stabilizing effect, glycerol-containing stream or the second stream fed to evaporation comprises from 5 to 90%-wt, preferably 5-83%-wt, more preferably 5-50%-wt and most preferably 5-30%-wt water of the total mass of glycerol-containing stream or the second stream respectively. The amount of water in glycerol-containing stream is dependent on the source and composition thereof, wherein components other than water and glycerol may be present as impurities dependent on the preceding processing or the origin of the stream. In some embodiments, water may be added to the glycerol containing stream.

On one hand, when the amount of water in the glycerol containing feed exceeds that needed for the vapor phase conversion, the water remaining in the liquid phase contributes as solvent for heavy impurities present in the feed. This aqueous phase with heavy impurities therein may thus be discarded. Thereby, no additional purification step is needed prior to conversion. Depending on the origin of the glycerol feed, said heavy impurities vary to some degree. Typical heavy impurities present in glycerol streams or crude glycerol from transesterification comprise at least trace amounts of unconverted mono and diglycerides and water-soluble Na-soaps. The formation of these is due to the base catalyst, such as NaOCH₄ used in transesterification processes. In case the glycerol stream originates from hydrolysis, it may contain some mono and diglycerides or polyglycerols, i.e. glycerol polyethers. Presence of soap type impurities or phospholipids is also possible.

Since the evaporation provides vaporization of glycerol and some water, the heavy impurities entering the reactor with the glycerol-containing stream or the second stream are retained at least partly, preferably at least 80% by weight, more preferably at least 95% by weight of the total weight of the impurities, or even totally, in the liquid aqueous phase and do not proceed to the conversion reactor. Considering the water entering evaporation, it is divided between the vapor and liquid phases, each contributing to advantageous effects; water in vapor phase stabilizes glycerol and water in the liquid phase dissolves and provides a matrix for removal of heavy impurities.

The inventors have found that the present process enables avoidance of purification steps of the glycerol before the conversion. Feeding a crude glycerol directly to the evaporator provides optimal conditions leaving heavy impurities to the liquid aqueous phase. Thus, light impurities, such as methanol, are evaporated and follow glycerol to the catalytic conversion step, whereas heavy impurities remain in the liquid aqueous phase. Therefore, according to one embodiment, an aqueous residue is withdrawn from the evaporation.

According to an embodiment, the glycerol-containing stream, which may be described also as a water-glycerol mixture, is heated at pressurized conditions. Water-glycerol mixture is entering into the catalyst bed comprising a catalyst able to convert glycerol to propanol. The catalytic glycerol conversion is conducted at a temperature below 400° C., preferably from 200° C. to 300° C., more preferably from 230° C. to 290° C., most preferably form 250° C. to 280° C. Preferably, the reaction is carried out at hydrogen pressure of about from 0.2 to 1.5 MPa, more preferably from 0.5 to 1.0 MPa. The pressure conditions ensure that the reaction takes place at gas phase.

The catalysts used in the catalytic glycerol conversion may be selected from metal catalysts on an acid support. The support material is selected from acidic inorganic materials stable at a temperature below 350° C. Catalysts may preferably be selected form those comprising a metal selected from Pt, Pd, Ni, Cr, Mo, W, Ru, Rh, Ir, Cu on acid oxide support. Noble metal based catalysts present good selectivity to propanols for hydrogenolysis of glycerol. The support may be selected from TiO₂, ZrO₂, WO₂, CrO₃, preferably TiO₂, ZrO₂, or modified TiO₂, ZrO₂, such as W-modified TiO₂ or ZrO₂.

According to a preferred embodiment, high conversion of glycerol, at least 95%, preferably 98%, more preferably 99.9%, and selectivity to propanols at least 85%, preferably 87%, more preferably 88%, may be achieved. The hydroprocessing of glycerol into propanols requires reduction at two of three hydroxyl groups. Hence, the rest of the converted glycerol is reacted to side products or compounds showing uncomplete reduction, such as acetol, acetone and diols, 1,2-propanediol and 1,3-propanediol. Diols may be recycled back to conversion reactor.

The present inventors have found that in the present conversion, both 1- and 2-propanols are produced so that the ratio of 2-propanol to 1-propanol ranges from 0.15 to 0.99, preferably from 0.2-0.6. Traces of different side products, such as methanol, ethanol, acetone, 1,2-propanediol, 1,3-propanidiol, may be present. Considering the renewable propanol gasoline component obtained from said process, it comprises from 14 to 49%-wt, preferably from 17 to 35%-wt 2-propanol, from 50 to 85%-wt, preferably from 65 to 83 wt-% 1-propanol and from 0.2 to 5 wt-% of an impurity selected from methanol, ethanol, acetol, acetone, 1,2-propanediol, 1,3-propanidiol and mixtures thereof and wherein sum of 2-propanol, 1-propanol and impurities adds up to 100%-wt of the renewable propanol gasoline component weight. Alternatively, in case of lower glycerol conversion, unreacted glycerol contributes to propanol recovery. In this case, unreacted glycerol may be recycled from propanol recovery back to conversion.

The product from catalytic glycerol conversion is fed to a separation and recovery step. Separation is conducted at a separation system comprising several unit operations. The product stream recovered contains the desired products 1-propanol and 2-propanol, a small amount of diols, such as 1,2-propanediol and some unreacted water and glycerol, and reactant hydrogen.

According to an embodiment, a pressure at the conversion of glycerol to propanol(s) between 0.2 and 1.5 MPa has also been found to provide a further benefit with regard to separation following the conversion. The pressurized conditions provide synergistic advantages when both the process of the catalytic glycerol conversion and at least one following separation step for recovery of 1-propanol, 2-propanol or a mixture thereof are conducted at a pressure between 0.2 and 1.5 MPa. When the separation for recovery of 1-propanol, 2-propanol or a mixture thereof, is conducted at pressure between 0.2 and 1.5 MPa, and preferably at a pressure essentially equal to that at the conversion, losses relating to pressure changes can be minimized or avoided. From the outlet of the conversion reactor, the product stream may be led to the separation unit without need for pressurization equipment or controls thereto.

The separation step following glycerol conversion may contain various steps. The first separation may be a gas-liquid separation unit that separates gaseous hydrogen from a liquid stream. The separation unit for the liquid stream can be distillation. According to a preferred embodiment the separation step comprises at least one distillation. In the system propanols-water-glycerol, the affinity of the glycerol for the water prevents the distillation of an alcohol-water azeotrope, and anhydrous propanols can be distilled out from the mixture.

According to another embodiment, the use of entrainers in distillation contributes to enhanced separability. Hygroscopic salts like potassium carbonate are known to make the distillation process more efficient. The unreacted glycerol may optionally be recycled back to glycerol feed stream. The hydrogen after the separation may also be optionally recycled to the make-up hydrogen.

According to another embodiment, another means for recovery of propanols after conversion may involve a hydrocarbon for splitting the azeotrope, such as cyclohexane, which hydrocarbon may leave a trace to the renewable propanol gasoline component.

Should the propanediols, 1-propanol and 2-propanol be separated from one another, at least a second distillation step will be required. The propanol fraction from the second distillation does not require further purification to be useful as a renewable propanol gasoline component.

The produced propanol fraction may be blended with renewable naphtha or gasoline components consisting of C4-C9-alkanes to produce a 100% biogasoline composition fulfilling the requirements of gasoline specifications. Such blends may be provided by the present process further comprising blending the renewable paraffinic naphtha component, such as renewable naphtha components obtained from the overall process (step e) with the renewable propanol gasoline component, i.e. providing a composition comprising a renewable propanol gasoline component with a renewable paraffinic naphtha component. It is noted that by the expression “renewable propanol gasoline component” refers herein to those gasoline components that originate from propanol production process as described above. Blending with other biobased gasoline components is also advantageous.

The renewable propanol gasoline component or a composition comprising both a renewable propanol gasoline component and a renewable paraffinic naphtha component, can also be used as a blending component for the state of art gasoline, such as gasoline obtained from fossil crudes. As used herein, “a fossil gasoline component” is intended to mean any mixture of organic compounds, said mixture having a boiling point in the range from e.g. about 30° C. to about 230° C., preferably from about 30° C. to about 210° C. Typically a fossil gasoline component is a combination of hydrocarbons comprising paraffins, and aromatic and olefinic hydrocarbons, having from 4 to 9 carbon atoms, wherein the olefinic content may be about vol % and the aromatic content about 40 vol %.

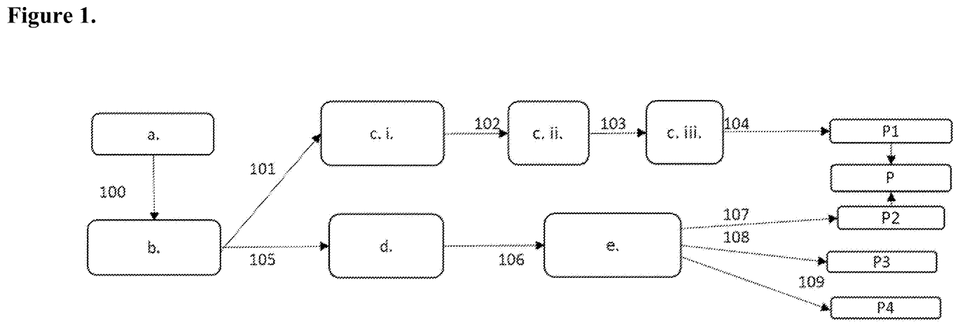

The present inventors have found that as a part of the combined fuel component production, the present process enables avoidance of purification steps of the glycerol before the catalytic conversion. Said combined production is depicted in schematic FIG. 1 with references to process steps (a., b. etc.). Feeding a crude glycerol, optionally with added water, directly to the evaporator provides optimal feed leaving impurities to the liquid phase. Therefore, the overall process for producing renewable fuel components comprises the steps of
a. providing a glyceride containing feedstock; and
b. splitting said glyceride containing feedstock to provide a first stream comprising fatty acids, or esters of fatty acids, and a second stream comprising glycerol and water; and
c. subjecting said second stream obtained from step b, and optionally added water to
   i. at least one evaporation in the presence of 5-90%-wt water of the total second stream weight, wherefrom the vapor phase is directed to;
   ii. catalytic conversion of glycerol to 1-propanol, 2-propanol or a mixture thereof at vapor phase in presence of water and hydrogen, and
   iii. separation and recovery of 1-propanol, 2-propanol or a mixture thereof as a renewable propanol gasoline component;
d. subjecting said first stream to hydroprocessing, to provide a first product stream of renewable paraffinic fuel components comprising i-paraffins and n-paraffins, and
e. separating said first product stream, and recovering renewable fuel components comprising at least one renewable paraffinic naphtha component.

Said process provides very efficient use of the glyceride containing feedstock material. Compared to prior art processes, wherein triglycerides are fed to hydroprocessing reactions, the glyceryl-component of triglycerides is not converted to propane, but may be converted to more valuable components, such as propanols suitable for use as biogasoline components.

### The Glyceride Containing Feedstock

The glyceride containing feedstock suitable for use according to the present invention comprises free fatty acids and glycerides, at least 5%, preferably at least 50%, more preferably at least 80% by weight glycerides of the total glyceride containing feedstock weight. Particularly suitable glyceride containing feedstocks for renewable paraffinic fuel component and, especially, paraffinic renewable base oil production, are those which comprise glycerides releasing abundantly C16 fatty acids in splitting, such as hydrolysis.

Several oils and fats contain significant amounts of C16 fatty acids (FA). Part of the fatty acids are already in the form of free fatty acids (FFA), but part are bound to glycerides as esters.

Table 2 lists availability of C16 and C18 free fatty acids, and the fatty acid carbon chain lengths and unsaturation of exemplary fats and oils found in the literature, possibly suitable for use in the process of the present invention.

TABLE 2
Exemplary glyceride containing feedstocks suitable for the process for producing
renewable fuel components and optionally paraffinic renewable base oil of the present
invention.

| | | | | | | | | | | | | | | Amount |
|---|---|---|---|---|---|---|---|---|---|---|---|---|---|---|
| | | | | | | | | | | | | | | of FFAs |
| | | | | | | | | | | | | | | ²Amount |
| | The fatty acid distribution of glyceride containing feedstocks suitable for the present process | | | | | | | | | | | | | of C16 |
| Fat/oil | (%-wt) | | | | | | | | | | | | | and C18 |
| (%) | 8:0 | 10:0 | 12:0 | 14:0 | 16:0 | 18:0 | 18:1 | 18:2 | 18:3 | 20:0 | 20:1 | 22:0 | 22:1 | FFAs |
| Canola | | | | 0.1 | 4.1 | 1.8 | 60.9 | 21.0 | | 0.7 | | 0.3 | | |
| Crude tall oil | | | | | ¹1-2 | | | | | | | | | |
| Cottonseed | | | | 0.7 | 21.6 | 2.6 | 18.6 | 54.4 | 0.7 | 0.3 | | 0.2 | | |
| Crumbe | | | | | 1.7 | 0.8 | 16.1 | 8.2 | 2.9 | 3.3 | | 2.2 | 59.5 | |
| Cuphea | 0.8 | 81.9 | 3.2 | 4.3 | 3.7 | 0.3 | 3.6 | 2.0 | 0.3 | | | | | |
| (PSR-23) | | | | | | | | | | | | | | |
| Jatropha | | | | | ¹15 | | | | | | | | | ¹1.5-5 |
| Palm | | | 0.2 | 1.1 | 44.0 | 4.5 | 39.1 | 10.1 | 0.4 | 0.4 | | | | ¹4-7 |
| Palm Kernel | 3.3 | 3.4 | 48.2 | 16.2 | 8.4 | 2.5 | 15.3 | 2.3 | | 0.1 | 0.1 | | | |
| Palm stearin | | | | | ¹60 | | | | | | | | | ¹0.1 |
| PFAD | | | | | ¹45 | | | | | | | | | ¹75-88 |
| Rapeseed | | | | | 2.7 | 1.1 | 14.9 | 10.1 | 5.1 | 10.9 | | 0.7 | 49.8 | |
| Soybean | | | 0.1 | 0.2 | 10.7 | 3.9 | 22.8 | 50.8 | 6.8 | 0.2 | | | | ¹2.5 |
| Sunflower | | | | | 3.7 | 5.4 | 81.3 | 9.0 | | 0.4 | | | | ¹0.5 |
| Lard | | 0.1 | 0.1 | 1.5 | 26.0 | 13.5 | 43.9 | 9.5 | 0.4 | 0.2 | 0.7 | | | ¹5-10 |
| Tallow | | | 0.1 | 3.2 | 23.4 | 18.6 | 42.6 | 2.6 | 0.7 | 0.2 | 0.3 | | | ¹5-10 |

¹Values measure at the Analytics lab of Neste Oyj by GC.
²Estimation of C16-C18 FFAs in % is based on ½ * TAN (total acid number analysis), which is a fair approximation.

Typical basic structural unit of plant and fish oils and animal fats is a triglyceride. Triglyceride is an ester of glycerol with three fatty acid molecules having the structure below:

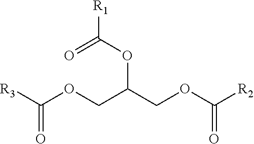

wherein R₁, R₂ and R₃ are same or different and represent saturated or unsaturated C3-C27 hydrocarbon chains. The length of the hydrocarbon chain for Rₓ is typically 17 carbons and hence splitting releases C18 fatty acids. Another typical length of the hydrocarbon chain for Rₓ is 15 carbons and hence splitting releases C16 fatty acids. In general, typical carbon numbers of the fatty acids linked to the two other hydroxyl groups are even, being generally between carbon chain lengths from C12 to C22.

In addition to the prevailing triglycerides, some diglycerides and monoglycerides are present as well. Diglycerides are esters of glycerol with two fatty acid molecules having alkyl group Rₓ (Rₓ—CO—) and monoglycerides are ester of glycerol with one fatty acid molecules having alkyl group Rₓ (Rₓ—CO—) bound therein. With reference to structure above, the number of substituents R, is 1, 2 or 3. These mono- and diglycerides release glycerol in hydrolysis as well. Mono- and diglycerides are formed in minor amounts spontaneously from triglycerides during storage or under pretreatment conditions, releasing some free fatty acids. Hence, the term “glyceride containing feedstock” refers to feed comprising mono-, di-, and triglycerides and free fatty acids.

Prior to processing, the glyceride containing feedstock of biological origin may be pretreated with suitable known methods, such as thermally, mechanically for instance by means of shear force, chemically for instance with acids or bases, or physically with radiation, distillation, cooling, or filtering. The purpose of chemical and physical pretreatments is to remove impurities interfering with the process or poisoning the catalysts, and to reduce unwanted side reactions. Hence, according to one embodiment, the glyceride containing feedstock is subjected to purification before entering into the splitting, such as hydrolysis step. This purification may include e.g. bleaching and/or deodorizing.

Thus, glyceride containing feedstocks suitable for the process of the present invention comprise mono- di- and/or triglycerides and free fatty acids. Exemplary glyceride containing feedstocks are plant fats, plant oils, plant waxes, animal fats, such as lard, tallow, yellow grease, brown grease, animal oils, animal waxes, fish fats, fish oils, and fish waxes. Preferably, the glyceride containing feedstock material originates from waste and/or residues of the mentioned exemplary glyceride containing feedstocks. More preferably, the waste and/or residues originate from sustainably-produced products, the production routes of which are traceable. Preferable feedstocks of animal origin are discussed in detail by Alm, M, (2013) Animal fats. [online]. Available at http://lipidlibrary.aocs.org/OilsFats/content.cfm?ItemNumber=40320 [Accessed 21.12.2018]. When the appropriate glyceride containing feedstock, optionally with pretreatment, is provided in step a., the next step, step b. cleaves the fatty acids from the glycerol backbone. Preferably, the fatty acid group may be cleaved without a chemical change to the carbon backbones.

### Splitting

As used herein, splitting is used to refer to reactions releasing glycerol from glyceride containing feedstock. Such reactions comprise saponification, hydrolysis and transesterification of glycerides. After splitting, the first and second streams, i.e. fatty acids or fatty acid esters and glycerol are obtained by separation of the splitting product.

### Saponification

Saponification is a reaction between a base, such as NaOH and triglyceride. The ester bond(s) are cleaved producing alcohols (here glycerol) and soaps of carboxylic acid(s), such as Na-soaps, which may also referred to as salts. These soaps of fatty acids are acidified before they can be reacted further in the present process. Acidification transfers the salt back to acid, the fatty acid. Hence, in case of splitting conducted as saponification, said first stream obtained from step b comprises fatty acids and is subjected to hydroprocessing in step d. According to a specific embodiment comprising ketonisation reaction in step d′, said fatty acids obtained through saponification followed by acidification are separated in a separation step b′ before step d′.

### Transesterification

Transesterification is a process well known in the art, i.e. for production of biodiesels. Glycerides are reacted in the presence of an alcohol to fatty acid esters. The most common alcohol is methanol, producing fatty acid methyl esters (FAME). If ethanol is used in transesterification, fatty acid ethyl esters (FAEE) are obtained. Hence, the ester bonds between glycerol and fatty acids are cleaved releasing glycerol, but the fatty acids are still in form of esters. The separation of glycerol from fatty acid esters formed is known in the art. During transesterification and downstream processing thereof, some water is accumulated to the glycerol stream. However, to the process including evaporation according to the present glycerol conversion process, addition of water is needed.

Hence, in case of splitting conducted as transesterification, said first stream obtained from step b comprises fatty acids esters and is subjected to hydroprocessing in step d. According to a specific embodiment comprising ketonisation reaction in step d′, said fatty acids esters are separated in a separation step b′ before step d′.

### Hydrolysis of Glyceride Containing Feedstock

Hydrolysis in the glycerides cleaves the ester bond(s) and produces an alcohol (here glycerol) and carboxylic acid(s) (here fatty acids).

According to a preferred embodiment, the splitting (step b) is conducted as hydrolysis. Hydrolysing the glyceride containing feedstock provides then a first stream comprising fatty acids, and a second stream comprising glycerol and water.

Hydrolysis can be carried out by refluxing the glyceride containing feedstock with different catalysts. The reactions catalyzed by acid, base, or lipase are known in the art. Hydrolysis is also known to occur as an un-catalyzed reaction between fats and water dissolved in the fat phase at suitable temperatures and pressures.

A hydrolysis may be performed in a hydrolysis unit using known methods, for example such as the commercial Colgate—Emery process or modifications thereof in a conventional manner described in literature in the art. The hydrolysis step produces a free fatty acid stream and an aqueous glycerol stream.

According to an exemplary embodiment purified palm oil as the glyceride containing feedstock, is fed from the bottom of a hydrolysis column, and water is fed from the top of the column. The high temperature, such as about 250° C., and high pressure, such as about 50 MPa, enhance the solubility of water in oil phase where hydrolysis of the glyceride containing feedstock takes place. The glyceride containing feedstock passes as a coherent phase from the bottom to the top through the hydrolysis column tower, whereas the heavier water travels downward as a dispersed phase through the mixture of oil and fatty acids. The mixture of fatty acid and entrained water is obtained at the top while the sweet water which contains from 10 to 18% of glycerol in water is recovered at the bottom. Approximately two hours of reaction time is needed to reach degree of hydrolysis up to 99%. The fatty acids are discharged from the top of the hydrolysis column to an evaporator, where the entrained water is separated or flashed off. The aqueous glycerol stream is removed to prevent oxidation and degradation of the fatty acids. The water vapor is then condensed and collected at a feed water tank.

According to another embodiment, the glyceride containing feedstock is hydrolyzed by base, such as sodium hydroxide, in a conventional manner described in literature in the art. The process produces glycerol and salts of fatty acids. The fatty acids are liberated from the salts prior to further processing by contacting them with strong mineral acids, such as sulfuric acid. Excess sulfuric acid and the formed sodium or potassium sulfate are removed by washing with water.

The hydrolysis unit comprises equipment materials which are suitable for acidic or corrosive reagents.

According to a specific embodiment, the hydrolysis is base catalyzed, and CO₂ produced in the ketonisation of fatty acid or fatty acid esters according to a specific embodiment of the present invention, could be used in the neutralization of base of hydrolysis process.

After cleavage of the fatty acids from the glycerol, the glycerol may be separated from the fatty acids. The separation may be accomplished by any suitable methods including liquid-liquid extraction, supercritical solvent extraction; distillation, membrane filtration, acidulation, centrifugation, by gravity separation, or combinations thereof. Once separated from the fatty acids, the second stream comprising glycerol is catalytically converted to products comprising propanols.

In embodiments, where the splitting is conducted as hydrolysis, the first stream of fatty acids obtained from hydrolysis is then subjected in step d. to hydroprocessing, such as hydrodeoxygenation, hydroisomerization or a combination thereof, to provide a first product stream of renewable paraffinic fuel components comprising i-paraffins and n-paraffins in the fuel or base oil range.

### Hydroprocessing

Hydroprocessing refers to hydrodeoxygenation, hydrodesulfurization, hydrodenitrogenation, hydrodehalogenation (such as hydrodechlorination) hydrogenation of double bonds, hydrocracking and/or hydroisomerisation and it also removes some metals. Within the context of the present process, hydroprocessing is needed for removal of covalently bound oxygen from the fatty acid and fatty acid ester molecules. Typically, this means deoxygenation by hydrogenation i.e. hydrodeoxygenation (HDO) and hydrogenation of double bonds. In the context of the present process, hydroprocessing of step d, or d′, or both d and d′, comprises hydrodeoxygenation. In addition or alternatively, hydroprocessing of step d, or d′, or both d and d′, comprises hydroisomerization. Preferably, any hydroprocessing comprises both hydrodeoxygenation and hydroisomerization.

### Hydrodeoxygenation

Hydrodeoxygenation (HDO) of the fatty acids may be carried out as depicted e.g. in FI100248B EP1741768A1, WO2007068795A1, WO2016062868A1 or EP2155838B1, and using a conventional hydroprocessing catalysts and hydrogen gas.

In one embodiment the hydrodeoxygenation takes place at reaction conditions comprising a temperature in the range from 100 to 500° C., preferably from 250 to 400° C., more preferably from 280-350° C., most preferably at temperature of 300-330° C.; and at a pressure in the range from 0.1 to 20 MPa, preferably from 0.2 to 8 MPa. Preferably, the weight hourly space velocity (WHSV) is in the range from 0.5 to 3.0 h⁻¹, more preferably from 1.0 to 2.5 h⁻¹, most preferably from 1.0 to 2.0 h⁻¹. Preferably, H₂ flow is in the range from 350 to 900 nl H₂/l feed, more preferably from 350 to 750, most preferably from 350 to 500, wherein nl H₂/l means normal liters of hydrogen per liter of the feed into the HDO reactor, in the presence of a hydrodeoxygenation catalyst. The hydrodeoxygenation catalyst is preferably selected from Pd, Pt, Ni, Co, Mo, Ru, Rh, W, or any combination of these, such as CoMo, NiMo, NiW, CoNiMo on a support, wherein the support is preferably alumina and/or silica.

According to one embodiment, the fatty acids may be subjected to both hydrodeoxygenation (HDO) reaction conditions and to hydroisomerization reaction conditions, simultaneously or in sequence, to yield a deoxygenated and isomerized paraffinic product stream comprising the fuel components.

### Isomerization (Hydroisomerization)

Isomerization can be carried out in a conventional hydroisomerization unit, such as those depicted in FI100248B, EP1741768A1, WO2007068795A1, WO2016062868A1 or EP2155838B1. Hydrogen is added into the isomerization step.

Both the hydrodeoxygenation step and hydroisomerization step may be conducted in the same reactor, and even in the same reactor bed. The hydroisomerization catalyst may be a noble metal bifunctional catalyst such as a Pt containing commercial catalyst, for example Pt-SAPO or Pt-ZSM-catalyst or for example a non-noble catalyst, such as NiW. The hydrodeoxygenation and hydroisomerization steps may be performed using NiW catalyst, or even in the same catalyst bed using the NiW catalyst for both the hydrodeoxygenation and isomerization. The NiW catalyst may additionally result in more hydrocracking to diesel and naphtha products.

The hydroisomerization step is preferably performed at a temperature from 250 to 400° C., more preferably from 280 to 370° C., most preferably from 300 to 350° C. Pressure is preferably from 1 to 6 M Pa, more preferably from 2 to 5 M Pa, most preferably from 2.5 to 4.5 MPa. The WHSV is preferably from 0.5 to 3 1/h, more preferably from 0.5 to 2 1/h, most preferably from 0.5 to 1 1/h, and H₂ flow from 100 to 800, more preferably from 200 to 650, most preferably from 350 to 500 n-liter Hz/liter feed, wherein n-liter H₂/l means normal liters of hydrogen per liter of the feed into the isomerization reactor.

During hydroisomerization n-paraffins are branched i.e. forming i-paraffins. Preferably, the conditions are chosen such that the branches are located at or near the terminal ends of the molecules, and therefore the cold flow properties of renewable base oil or renewable fuels are improved.

During the conventional hydroisomerization of n-paraffins to fuel components some cracking may be present. Therefore, the selection of the catalyst and optimization of reaction conditions are always important during the isomerization step. Due to cracking during isomerization renewable diesel and naphtha are formed, and may even be formed from longer carbon chain length n-paraffins such as those of renewable base oil. The renewable diesel component thus obtained has typically excellent cold flow properties and can be used as winter grade diesel fuel as is i.e. 100%, without blending it to fossil middle distillate.

As the last step of the process, said first product stream is separated, and renewable fuel components recovered comprising at least one renewable paraffinic naphtha component. The products may be fractionated using conventional separation processes, especially fractionation by vacuum distillation, which can separate the branched paraffinic product mixture into renewable liquefied petroleum gas (LPG) comprising C3 and C4 hydrocarbon components; renewable naphtha suitable for use as gasoline component; renewable diesel fuel and/or aviation fuel i.e. aviation fuel such as HEFA or HEFA+ components, transformer oil components such as transformer oil having a boiling point of 280-300° C. or alternatively 280-350° C.; and to renewable base oil.

As a specific embodiment of the process of the invention, the catalytic conversion of glycerol to 1- and/or 2-propanol relates to combined fuel component and base oil production. The present process then comprises
a. providing a glyceride containing feedstock; and
b. splitting said glyceride containing feedstock to provide a first stream comprising fatty acids or fatty acid esters, and a second stream comprising glycerol; and
b′. separating the first stream obtained from step b into at least two fatty acid fractions, fraction 1 and fraction 2, and
c. subjecting said second stream obtained of step b, and optionally added water to
   i. at least one evaporation in the presence of 5-90%-wt water of the total stream weight, wherefrom the vapor phase is directed to;
   ii. catalytic conversion of glycerol to 1-propanol, 2-propanol or a mixture thereof at vapor phase in presence of water and hydrogen, and
   iii. separation and recovery of 1-propanol, 2-propanol or a mixture thereof as a renewable propanol gasoline component;
d. subjecting fraction 1 to hydroprocessing to provide a first product stream of renewable paraffinic fuel components comprising i-paraffins and n-paraffins;
d′. subjecting fraction 2 to ketonisation prior to hydroprocessing to provide a second product stream of renewable base oil comprising i-paraffins and n-paraffins; and
e. separating and recovering from the second product stream or combined first and second product streams, renewable fuel component(s) comprising at least one renewable paraffinic naphtha component.

In an embodiment, where the process proceeds through separation (in step b′ above), said separation comprises at least one distillation. Distillation provides effective means for division of the first stream comprising fatty acids into at least two fractions. Distillation provides at least a fraction 1 and a fraction 2. According to an embodiment, fatty acids are recovered in different fractions based on their carbon chain lengths.

The non-volatile impurities in the distillation bottom can be removed using conventional methods, such as degumming and/or bleaching.

In said separating the first stream, the separating can be done in a single distillation step or in two or three or more distillation steps. The distillation further purifies the distillate streams from metals and other heavy impurities which will reside after distillation at the bottom fraction. The first stream comprising fatty acids and separated from the second stream comprising glycerol by hydrolysis remain pure due to the impurities remaining in the glycerol phase. In an embodiment where palm oil is used as the glyceride containing feedstock to the overall process for producing renewable fuel components, the fatty acid distribution after hydrolysis follows that of said glyceride containing feedstock. The predominant fatty acids are oleic acid (C18:1) and palmitic acid (016:0). Accordingly, more than 70%-wt of the total weight of fraction 1 consists of C18-fatty acids or fatty acid esters. Further, 70%-wt of the total weight of fraction 2 consists of C16-fatty acids or fatty acid esters.

According to a specific embodiment, said distillation provides three distillation cuts. The fraction comprising fatty acids or fatty acid esters having the carbon chain length between C12 and C16 is referred here as fraction 2. The two other cuts, the lightest cut comprising fatty acids or fatty acid esters having a carbon chain length of C11 or less and the heaviest fraction comprising the fatty acids or fatty acid ester having carbon chain length of C17 or more are referred here together as fraction 1.

The separation may be realized by using at least one vacuum distillation column, preferably from two to four columns, which may be in series, depending on the accuracy needed for the separation and on the fatty acid distribution of the glyceride containing feedstock, the glyceride containing feedstock type and quality.

According to one exemplary embodiment of the separating of fatty acids, said separation produces
a fatty acid fraction wherein at least 90% of the fatty acids have a carbon chain length of C11 or less i.e. an optional effluent stream (v) boiling below 260° C., preferably below 240° C., at atmospheric pressure.
a fatty acid fraction 2 wherein at least 90% of the fatty acids have a carbon chain length from C12 to C16 i.e. the effluent stream (iii) boiling at a range from 260° C. to 360° C., preferably at a range from 298° C. to 352° C., at atmospheric pressure.
a fatty acid fraction wherein at least 90% of the fatty acids have a carbon chain length of C17 or more, i.e. an optional effluent stream (iv) boiling above 360° C., preferably above 374° C., at atmospheric pressure,
   and a further fraction comprising the eventual remaining glycerides i.e the distillation bottom.

The distillation temperatures are typically those measured at the exit of the distillation column(s). Herein the distillation temperatures are mathematically scaled to atmospheric pressures.

Renewable base oil may be produced from the fatty acids, preferably from saturated fatty acids or esters containing a high content of C16 hydrocarbons. Preferably, the feed is first ketonised, hydrodeoxygenized and/or isomerized as described for the fuel production.

Ketonisation reaction is an excellent deoxygenation reaction when deoxygenation, stability and energy density of products are the targets, as is often the case in production of fuels and base oils. Ketonisation removes 75 mol-% of the oxygen bound to carboxylic acid molecules without hydrogen. This is very important for fuel applications aiming at greenhouse gas (GHG) emission reduction. During the ketonisation reaction two fatty acid molecules are reacted together forming the corresponding linear ketone. One molecule of CO₂ and water is simultaneously released during the reaction.

Ketonisation reaction can be carried out with high conversion, such as 95%, or 98%, or even 99.9%, and with excellent selectivity, such as 85%, or 92%, or even 95%, which is the reason why the renewable base oil yield can be almost theoretical. Due to the very selective ketonisation reaction only few or no light hydrocarbons are formed, therefore, bio-CO₂ recovered from the ketonisation reaction can be very pure, preferably at least 99% by volume, and it can be used for varying applications. Naturally, the ketones produced from the free fatty acid fractions obtained by the process of the present invention may also be used as chemicals for various applications other than base oil or fuel component production.

Ketonisation conditions are typically specified by the reactor temperature and pressure, the used catalyst, the carrier gas/feed ratio and weight hourly space velocity of the feed. The selected ranges may be combined according to need depending on the parameters to be optimized.

In the present invention, the ketonisation reaction may be carried out at a reaction temperature ranging from 100 to 500° C., preferably from 300 to 400° C., more preferably from 330 to 370° C., most preferably from 340 to 360° C. The pressure range may be from atmospheric pressure to 10 MPa, preferably from 0.5 to 3.0 MPa, more preferably from 1.0 to 2.5 MPa, most preferably from 1.5 to 2.0 MPa, in the presence of a ketonisation catalyst. A suitable ketonisation catalyst comprises one or more metal oxide catalysts, preferably the metal of the metal oxide catalyst is selected from one or more of Na, Mg, K, Sc, Fe, Co, Ni, Cu, Zn, Sr, Y, Zr, Mo, Rh, Cd, Sn, La, Pb, Bi, Ti, Mn, Mg, Ca Zr and rare earth metals. More preferably, the ketonisation catalyst is a metal oxide catalyst selected from the list consisting of one or more of: Ti, Mn, Mg, Ca, and Zr containing metal oxide catalyst. Most preferably, the catalyst is Ti containing metal oxide catalyst, such as K₂O/TiO₂ catalyst, or TiO₂ containing catalyst, such as TiO₂ catalyst. The weight hourly space velocity (WHSV) may be in the range from 0.25 to 3.0 h-1, preferably from 0.5 to 2.0 h-1, more preferably from 1.0 to 1.5 h-1. Ketonisation reaction may be performed in the presence of a gas in the range from 0.1 to 1.5 gas/feed ratio (w/w), preferably from 0.25 to 1.0, most preferably from 0.5 to 0.75, wherein the gas/feed ratio (w/w) means the mass of gas fed into the ketonisation reactor per the inlet fatty acid mass of the liquid feed into the ketonisation reactor. The gas is selected from one or more of: CO₂, H₂, N₂, CH₄, H₂O. A particular gas is H₂, which may advantageously flow through the reactor into the next phase also requiring the presence of hydrogen, such as HDO. The most preferred gas is CO₂ as this is the product gas and may be efficiently recycled back to the feed, and it provides the most selective ketonisation reaction.

According to the embodiment comprising renewable base oil production through ketonisation step, the following hydroprocessing are preferably adapted to this particular stream. Preferably hydroprocessing is conducted as hydrodeoxygenation and hydroisomerization, either as a sequence or together in one step. It may be desirable to reduce the severity of the hydroisomerization reaction to avoid or to reduce the amount of cracking of the renewable base oil product by selecting suitable combinations from the temperature, pressure WHSV and H₂ flow ranges of temperature from 250 to 400° C.; pressure is from 1 to 6 Mpa; the WHSV is from 0.5 to 3 1/h; and H₂ flow n-liter H₂/liter feed from 100 to 800. However, cracking during hydroisomerization of the ketonised stream produces naphtha components, which when combined with renewable propanol gasoline component may provide the novel 100% renewable gasoline.

As the last step of the process said first and second product streams are separated, and renewable fuel components recovered comprising at least one renewable paraffinic naphtha component and a renewable base oil.

### Products

As one aspect of the present invention herein is provided a use of a glyceride containing feedstock for production of a renewable propanol gasoline component. Here, said production is combined with production of at least one renewable gasoline component, such as renewable naphtha components. Further renewable fuel components or renewable base oil or precursors thereto may be recovered from the process, as well.

The products obtainable by the present process can be characterized as renewable fuel components, such as diesel fuel components, naphtha components suitable for use in gasoline, aviation fuel components, and renewable base oil. Such components are used as such or in blends providing products fulfilling specifications set for said products.

The renewable content may be determined from both the starting materials and the products by isotopic distribution involving ¹⁴C, ¹³C and/or ¹²C as described in ASTM D6866.

With respect to the term “renewable” in the context of a renewable fuel component, such as renewable paraffinic naphtha component and renewable propanol gasoline component this term refers to one or more organic compounds derived from any renewable source (i.e. not from any fossil based source). Such component is characterised by mandatorily having a higher content of ¹⁴C isotopes than similar components derived from fossil sources. Said higher content of ¹⁴C isotopes is an inherent feature characterizing the renewable fuel component and distinguishing it from fossil fuels.

Any material of biological origin means material having about 100%-wt renewable (i.e. contemporary or biobased or biogenic) carbon, ¹⁴C, content which may be determined using radiocarbon analysis by the isotopic distribution involving ¹⁴C, ¹³C and/or ¹²C as described in ASTM D6866 (2018).

Products obtainable by the present process are used as components to products fulfilling standards set for respective products, such as renewable aviation fuel consisting of paraffinic hydrocarbons having carbon chain length from C6 to C17, fulfilling the ASTM D7566-16b or ASTM D7566-19, Annex A2 specification, having a density of less than 772 kg/m³ as measured according to ASTM 4052, and a freezing point of less than −40° C. as measured according to IP529, renewable diesel fuel consisting of paraffinic hydrocarbons fulfilling the EN 15940:2016, renewable gasoline fulfilling the EN228:2017 European standard specification requirements, and renewable base oil fulfilling the API Group III base oil specifications having ≥90 wt % saturated hydrocarbons, 0.03 wt-% sulfur and a viscosity index of ≥120.

The renewable propanol obtained from the present process is usable as a component for gasoline. Hence, herein is provided gasoline comprising a renewable propanol gasoline component. It is understood that the renewable propanol gasoline component comprises and preferably essentially consists of 1-propanol, 2-propanol or a mixture thereof. To provide desired characteristics, the composition comprises 1-propanol, 2-propanol or a mixture thereof in an amount from 6 to 15%-wt, preferably from 6 to 12%-wt of the total weight of the composition.

The renewable propanol gasoline component may be used to increase the biocontent of any gasoline. At minimum, the biocontent may be solely due to said renewable propanol gasoline component, wherein other components are non-renewable. Such a blend may be provided by blending fossil gasoline component, obtainable for example by distillation of crude oils, with renewable propanol gasoline component. However, if further components are used, they may be selected from renewable or non-renewable alternatives. According to some embodiments, the composition may further comprise renewable paraffinic naphtha component from 7 to 94%-wt, preferably from 10 to 94%-wt, more preferably from 85 to 94%-wt of the total gasoline weight. For example, ethanol is commercially available both as renewable and non-renewable qualities.

The hydrodeoxygenation, isomerization or combination thereof provides renewable paraffinic fuel components as products. The term “renewable paraffinic fuel component” defines said products being saturated hydrocarbons suitable for use as components for certain fuel grades. Paraffinic refers to the character as alkanes, straight chain or branched, not containing heteroatoms or double bonds. The expression “renewable paraffinic naphtha component” is herein taken to mean the naphtha components suitable for use as gasoline components or in gasoline blend compositions. Preferably, they are produced by the hydroprocessing, or by a combination of hydrodeoxygenation and isomerization route for fuel component production or by the ketonisation, hydrodeoxygenation, and isomerization route for RBO production as discussed above. In the present process renewable paraffinic naphtha component recovered in step e.

According to the invention, the renewable gasoline component may comprise essentially a mixture of C4-C9 hydrocarbons (i.e. hydrocarbons having 4 to 9 carbon atoms), such as a mixture of C4-C9 n-alkanes and iso-alkanes or a mixture of C4-C8 alkanes. Put differently, the renewable gasoline component may comprise a mixture of C4 to C9 hydrocarbons (CnH2n+2, n=4, 5, 6, 7, 8 or 9), i.e. straight or branched hydrocarbons having 4 to 9 carbon atoms originating from renewable sources such as e.g. plant or animal material and consequently not derived from any fossil based material. Non-limiting examples of relevant hydrocarbons may be n-alkanes and/or iso-alkanes. Consequently, the renewable gasoline component may comprise a mixture of one or more of n-hexane, n-pentane, 2-methylbutane (iso-pentane) and other C4 to C9 alkanes such as e.g. 2-methyl pentane, 2,3-dimethyl butane, heptane, 3-methyl hexane.

The renewable gasoline component may be very low in aromatic contents, i.e. contain a low amount of aromatic compounds such as e.g. benzene and/or toluene. Thus, the aromatic content may be e.g. about 0.1 vol %, preferably less than 0.1 vol % or even aromate-free.

The renewable gasoline component may have a boiling point range of e.g. about 40° C. to about 170° C.

Selecting a renewable paraffinic naphtha component to be blended with a renewable propanol gasoline component, a composition with a range up to 100% renewable is obtainable. In such a case, herein is provided a composition consisting of renewable 1-propanol, 2-propanol or a mixture thereof and a renewable paraffinic naphtha component. Hence, thereby gasoline can be provided consisting of renewable propanols and a renewable paraffinic naphtha component, wherein 100%-wt of the total gasoline weight is of renewable origin. To provide desired characteristics, the composition comprises 1-propanol, 2-propanol or a mixture thereof in an amount from 6 to 15%-wt, preferably from 6 to 12%-wt of the total weight of the composition. The proportion of the renewable paraffinic naphtha component may vary from 85 to 94%-wt of the total weight of the composition.

Said renewable paraffinic naphtha component comprises essentially a mixture of C4-C9 hydrocarbons, preferably a mixture of C4-C9 n-alkanes and isoalkanes. More specifically, the renewable paraffinic naphtha component may be characterized as comprising 10-40%-wt of a mixture of C4-C9 n-alkanes, 30-80%-wt of a mixture of C4-C9 isoalkanes, 2-15%-wt of a mixture of C4-C9 cycloalkanes, no more than 1%-wt of alkenes, no more than 1%-wt of aromatic hydrocarbons and no more than 0.5%-wt in total of oxygen containing compounds, wherein the total amount C4-C9 n-alkanes, C4-C9 isoalkanes and C4-C9 cycloalkanes is at least 95%-wt of the total renewable paraffinic naphtha component weight. According to some embodiments, 010 hydrocarbons may be present. Such renewable paraffinic naphtha component may have a boiling point in range of about 40° C. to about 170° C. at normal pressure.

In addition to gasoline components, at least one further component selected from, renewable paraffinic diesel component and renewable paraffinic aviation fuel component can be recovered in step e. Further, renewable base oil may be recovered from step e following step d′.

The present processes may be described with reference to Figures. FIG. 1 outlines as a schematic presentation the process of the present invention for producing renewable fuel components. A glyceride containing feedstock is provided in process step a. Stream 100 denotes feed of said glyceride containing feedstock to splitting reactor b. Splitting said glyceride containing feedstock provides a first stream 105 comprising fatty acids or esters of fatty acids, and a second stream comprising glycerol 101. Said second stream and additional water if needed, is in step c.i. subjected to at least one evaporation in the presence of water. The vapor phase therefrom 102, is directed to step c.ii, where catalytic conversion of glycerol to 1-propanol, 2-propanol or a mixture thereof takes place at vapor phase in presence of water and hydrogen (hydrogen feed not shown in figure). Stream 103 leads reaction products to separation and recovery, wherefrom stream 104 denotes product stream of 1-propanol, 2-propanol or a mixture thereof as a renewable propanol gasoline component P1.

Following the first stream (105) comprising fatty acids or esters of fatty acids, they are subjected to hydroprocessing in step d. The reaction product stream 106 is led to separation unit, wherefrom renewable fuel components, are divided to fractions 107, 108, 109, and recovered as renewable paraffinic fuel components, renewable paraffinic naphtha component P2, renewable paraffinic aviation fuel component, P3 and renewable paraffinic diesel fuel component P4, respectively.

Product streams P1 and P2 provide renewable components which can be blended to a 100% renewable gasoline, product P.

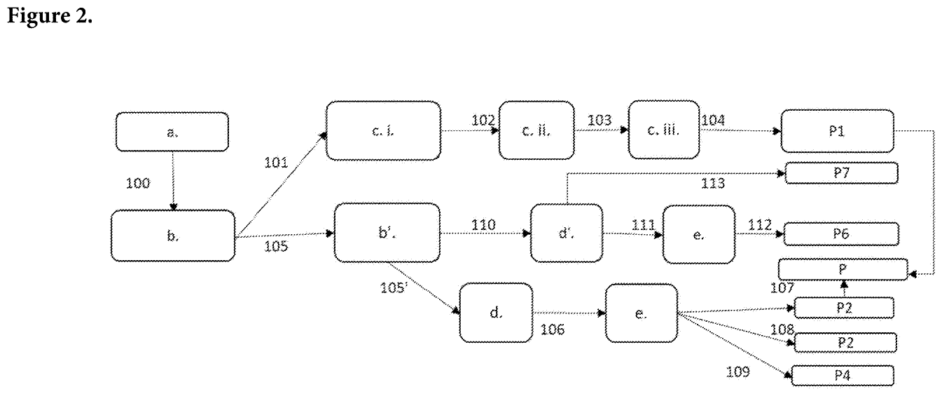

FIG. 2 outlines as a schematic presentation a preferred embodiment of the present invention for producing renewable fuel components and renewable base oil. A glyceride containing feedstock is provided in process step a. Stream 100 denotes feed of said glyceride containing feedstock to splitting, such as hydrolysis, in reactor b. Splitting said glyceride containing feedstock provides a first a second stream comprising glycerol 101. Said second stream, and additional water if needed, is in step c.i. subjected to at least one evaporation in the presence of water. The vapor phase therefrom 102 is directed to step c.ii, where catalytic conversion of glycerol to 1-propanol, 2-propanol or a mixture thereof takes place at vapor phase in presence of water and hydrogen (hydrogen feed not shown in figure). Stream 103 leads reaction products to separation and recovery, wherefrom stream 104 denotes product stream of 1-propanol, 2-propanol or a mixture thereof as a renewable propanol gasoline component P1.

The first stream (105) comprising fatty acids in now subjected to separation step b′. Through separation b′, at least two fractions. In FIG. 2, fraction 1 (110) is led to step d′ comprising ketonisation and hydroprocessing. Preferably the separation of step b′ is conducted to recover fatty acids within carbon chain length of C12-C16. Ketonisation produces stream 113 of renewable CO₂, which is recovered as product P7. Ketonisation product, typically having carbon chain length of C31 is subjected to hydroprocessing and the product stream 111 separated and recovered in step e giving product P6. From the separation b′ the fatty acids having carbon chain lengths of <C11 and >C17 are conducted to hydroprocessing in step d. wherefrom the reaction product stream 106 is led to separation unit e. Again, renewable fuel components, are divided to fractions 107, 108, 109, and recovered as renewable paraffinic fuel components, renewable paraffinic naphtha component P2, renewable paraffinic aviation fuel component, P3 and renewable paraffinc diesel fuel component P4, respectively.

Product streams P1 and P2 provide renewable components which can be blended to a 100% renewable gasoline, product P.

1. A process for producing renewable fuel components, said process comprising steps a.-e. of,
a. providing a glyceride containing feedstock; and
b. splitting said glyceride containing feedstock to provide a first stream containing at least one or more of fatty acids, or esters of fatty acids, and a second stream containing glycerol and water; and
c. subjecting said second stream obtained from step b, to:
i. at least one evaporation in a presence of 5-90%-wt water of a total second stream weight, wherefrom a vapor phase is directed to:
ii. catalytic conversion of glycerol to 1-propanol, 2-propanol or a mixture thereof at vapor phase in presence of water and hydrogen, and
iii. separation and recovery of 1-propanol, 2-propanol or a mixture thereof as a renewable propanol gasoline component;
d. subjecting said first stream to hydroprocessing, to provide a first product stream of renewable paraffinic fuel components containing i-paraffins and n-paraffins; and
e. separating said first product stream, and recovering renewable fuel components containing at least one renewable paraffinic naphtha component.

2. The process according to claim 1, for producing renewable fuel components, said process comprising:
b′. separating the first stream obtained from step b into at least two fatty acid or fatty acid ester fractions, fraction 1 and fraction 2; and
c. subjecting said second stream obtained of step b, and optionally added water to:
i. the at least one evaporation in the presence of 5-90%-wt water of the total second stream weight, wherefrom the vapor phase is directed to:
ii. catalytic conversion of glycerol to 1-propanol, 2-propanol or a mixture thereof at vapor phase in presence of water and hydrogen, and
iii. separation and recovery of 1-propanol, 2-propanol or a mixture thereof as a renewable propanol gasoline component;
d. subjecting fraction 1 to hydroprocessing to provide a first product stream of renewable paraffinic fuel components containing i-paraffins and n-paraffins;
d′. subjecting fraction 2 to ketonisation prior to hydroprocessing to provide a second product stream containing paraffinic renewable base oil containing i-paraffins and n-paraffins; and
e. separating and recovering from the second product stream or combined first and second product streams, renewable fuel component(s) containing at least one renewable paraffinic naphtha component.

3. The process according to claim 2, wherein the separation in step b′ comprises:
distillation.

4. The process according to claim 3, wherein more than 70%-wt of a total weight of fraction 1 consists of C18-fatty acids or fatty acid esters.

5. The process according to claim 4, wherein more than 70%-wt of a total weight of fraction 2 consists of C16-fatty acids or fatty acid esters.

6. The process according to claim 5, wherein an aqueous residue is withdrawn from the evaporation (i).

7. The process according to claim 6, wherein the catalytic glycerol conversion (ii) is conducted at a temperature selected to be at least one of below 400° C., and/or from 200° C. to 300° C., and/or from 230° C. to 290° C., and/or 250° C. to 280° C.

8. The process according to claim 2, wherein the catalytic glycerol conversion (ii) and at least one separation step (iii) for recovery of 1-propanol, 2-propanol or a mixture thereof are conducted at a pressure between 0.2 and 1.5 MPa.

9. The process according to claim 8, wherein the separation step (iii) comprises:
at least one distillation.

10. The process according to claim 2, wherein the hydroprocessing of step d, or d′, or of both d and d′, comprises:
hydrodeoxygenation.

11. The process according to claim 10, wherein the hydrodeoxygenation is conducted at reaction conditions comprising:
a temperature selected to be in a range from at least one or more of 100 to 500° C., and/or from 250 to 400° C., and/or from 280-350° C., and/or at temperature of 300-330° C.; and at a pressure in a range selected to be from 0.1 to 20 MPa, and/or from 0.2 to 8 MPa.

12. The process according to claim 10, wherein the hydrodeoxygenation is conducted at a weight hourly space velocity (WHSV) in a range selected to be from at least one or more of 0.5 to 3.0 h⁻¹, and/or from 1.0 to 2.5 h⁻¹, and/or from 1.0 to 2.0 h⁻¹.

13. The process according to claim 10, wherein the hydrodeoxygenation is conducted at H₂ flow in a range selected to be from at least one or more of 350 to 900 nl H₂/l feed, and/or from 350 to 750, and/or from 350 to 500, wherein nl H₂/l means normal liters of hydrogen per liter of feed into the HDO reactor, in a presence of a hydrodeoxygenation catalyst selected from Pd, Pt, Ni, Co, Mo, Ru, Rh, W, or any combination of these, including CoMo, NiMo, NiW, CoNiMo on a support, wherein the support is alumina and/or silica.

14. The process according to claim 2, wherein the hydroprocessing of step d, or d′, or both d and d′, comprising:
hydroisomerisation.

15. The process according to claim 14, wherein the hydroisomerization is performed at a temperature selected to be from at least one or more of 250 to 400° C., and/or from 280 to 370° C., and/or from 300 to 350° C., and at pressure selected to be from at least one or more of 1 to 6 MPa, and/or from 2 to 5 MPa, and/or from 2.5 to 4.5 MPa.

16. The process according to claim 14, wherein the hydroisomerization conducted at an WHSV is selected to be at least one or of from 0.5 to 3 1/h, and/or from 0.5 to 2 1/h, and/or from 0.5 to 1 1/h,

17. The process according to claim 14, wherein the hydroisomerization is conducted at H₂ flow selected to be from at least one or more of 100 to 800, and/or from 200 to 650, and/or from 350 to 500 n-liter H₂/liter feed, in a presence of an hydroisomerization catalyst selected from noble metal bifunctional catalysts, Pt containing commercial catalysts, and/or from Pt-SAPO or Pt-ZSM-catalyst or a non-noble catalyst, and/or NiW.

18. The process according to claim 10, wherein hydrodeoxygenation and hydroisomerization are performed simultaneously using NiW catalyst.

19. The process according to claim 2, wherein step e comprises:
recovery of at least one further component selected from renewable paraffinic diesel fuel component;
renewable paraffinic aviation fuel component; and
renewable transformer oil.

20. The process according to claim 2, comprising:
blending the renewable paraffinic naphtha component with the renewable propanol gasoline component.

21. A renewable propanol gasoline component comprising:
from 50 to 85%-wt 1-propanol, from 14 to 49%-wt 2-propanol, and from 0.2 to 5 wt-% an impurity selected from methanol, ethanol, acetol, acetone, 1,2-propanediol, 1,3-propanidiol and mixtures thereof, and wherein a sum of 2-propanol, 1-propanol and said impurities adds up to 100%-wt of the renewable propanol gasoline component weight and wherein a ratio of 2-propanol to 1-propanol is selected to be in a range from at least one or more of from 0.15 to 0.99, and/or from 0.2-0.6.

22. The component according to claim 21, comprising:
at least one or more of from 14 to 49%-wt, and/or from 17 to 35%-wt 2-propanol, from 50 to 85%-wt, and/or from 65 to 83 wt-% 1-propanol, and from 0.2 to 5 wt-% of an impurity selected from methanol, ethanol, acetol, acetone, 1,2-propanediol, 1,3-propanidiol and mixtures thereof and wherein a sum of 2-propanol, 1-propanol and said impurities adds up to 100%-wt of the renewable propanol gasoline component weight.

23. The component according to claim 22, comprising:
1-propanol, 2-propanol or a mixture thereof.

24. A composition comprising:
the renewable propanol gasoline component according to claim 21; and
a renewable paraffinic naphtha component.

25. The composition according to claim 24, wherein the renewable paraffinic naphtha component comprises:
the renewable paraffinic naphtha component recovered in step e.

26. The composition according to claim 24 comprising:
1-propanol, 2-propanol or a mixture thereof in an amount selected to be at least one or more of from 6 to 15%-wt, and/or from 6 to 12%-wt of a total weight of the composition.

27. The composition according to claim 24, comprising:
renewable paraffinic naphtha component selected to be at least one or more of from 7 to 94%-wt, and/or from 10 to 94%-wt, and/or from 85 to 94%-wt of a total weight of the composition.

28. The composition according to claim 24, wherein the renewable paraffinic naphtha component comprises:
essentially a mixture of C4-C9 hydrocarbons, and/or a mixture of C4-C9 n-alkanes and isoalkanes; or the renewable paraffinic naphtha component consists of C4-C8-alkanes, and/or a mixture of C4-C8 n-alkanes and isoalkanes.

29. The composition according to claim 24, wherein the renewable paraffinic naphtha component comprises:
10-40%-wt of a mixture of C4-C9 n-alkanes;
30-80%-wt of a mixture of C4-C9 isoalkanes;
2-15%-wt of a mixture of C4-C9 cycloalkanes, no more than 1%-wt of alkenes,
no more than 1%-wt of aromatic hydrocarbons; and
no more than 0.5%-wt in total of oxygen containing compounds, wherein a total amount C4-C9 n-alkanes, C4-C9 isoalkanes and C4-C9 cycloalkanes is at least 95%-wt of the total renewable paraffinic naphtha component weight.

30. The composition according to claim 24, wherein the renewable paraffinic naphtha component has a boiling point in range of about 40° C. to about 170° C. at normal pressure.

31. The composition according to claim 24, consisting of:
renewable 1-propanol, 2-propanol or a mixture thereof and a renewable paraffinic naphtha component, wherein 100%-wt of a total gasoline weight is of renewable origin.

32. The composition according to claim 24, comprising:
a fossil gasoline component.

33. The composition according to claim 32, wherein the fossil gasoline component comprises:
essentially a mixture of organic compounds, said mixture having a boiling point in the range selected to be at least one or more of from about 30° C. to about 230° C., and/or from about 30° C. to about 210° C.

34. The composition according to claim 32, wherein the fossil gasoline component comprises:
essentially a combination of hydrocarbons containing paraffinic, aromatic and olefinic hydrocarbons, having from 4 to 9 carbon atoms.

35. The composition according to claim 32, wherein the olefinic content is about 20 vol % and the aromatic content about 40 vol % of the fossil gasoline component.

36. Method for applying a renewable propanol gasoline component, the method comprising:
selecting a renewable proponol gasoline component containing from 50 to 85%-wt 1-propanol, from 14 to 49%-wt 2-propanol, and from 0.2 to 5 wt-% an impurity selected from methanol, ethanol, acetol, acetone, 1,2-propanediol, 1,3-propanidiol and mixtures thereof, and wherein a sum of 2-propanol, 1-propanol and said impurities adds up to 100%-wt of the renewable propanol gasoline component weight and wherein a ratio of 2-propanol to 1-propanol is selected to range from at least one or more of from 0.15 to 0.99, and/or from 0.2-0.6; and
producing a gasoline component with the renewable propanol gasoline component.

37. The method according to claim 36, comprising:
producing a combined fuel component with the gasoline component production.

38. A process for converting glycerol to renewable propanol gasoline component, containing:
from 50 to 85%-wt 1-propanol;
from 14 to 49%-wt 2-propanol; and
from 0.2 to 5 wt-% an impurity selected from methanol, ethanol, acetol, acetone, 1,2-propanediol, 1,3-propanediol, the process comprising:
a catalytic conversion at a vapor phase in a presence of water and hydrogen at a hydrogen pressure between 0.2 and 1.5 MPa, in a presence of a catalyst including metal selected from Pt, Pd, Ni, Cr, Mo, W, Ru, Rh, Ir, Cu on an acid oxide support, wherein a feed including glycerol and 5-90%-wt of water of the total feed weight, is subjected to evaporation wherefrom a vapor phase is conducted to said catalytic conversion.

39. The process according to claim 38, wherein an aqueous residue is withdrawn from the evaporation.

40. The process according to claim 38, wherein the conversion is conducted at a temperature selected to be at least one or more of from below 400° C., and/or from 200° C. to 300° C., and/or from 230° C. to 290° C., and/or from 250° C. to 280° C.

41. The process according to claim 38, comprising:
conducting at least one separation step following said conversion for recovery of 1-propanol, 2-propanol or a mixture thereof at a pressure between 0.2 and 1.5 MPa.

42. The process according to claim 41, comprising:
feed containing 5-50%-wt and/or 5-30%-wt of water of the total feed weight.

43. The process according to claim 41, wherein the glycerol is obtained from hydrolysis of triglycerides.

44. The process according to claim 43, wherein the feed comprises:
crude glycerol.
